(19) United States
(12) Patent Application Publication
(10) Pub. No.: US 2015/0038513 A1
(43) Pub. Date: Feb. 5, 2015

(54) ASSOCIATION BETWEEN N-HYDROXY-4-BENZAMIDE AND FOLFOX

(71) Applicants: LES LABORATOIRES SERVIER, Suresnes Cedex (FR); PHARMACYCLICS, INC., SUNNYVALE, CA (US)

(72) Inventors: Stéphane Depil, Issy Les Moulineaux (FR); Anne Jacquet-Bescond, Le Plessis Robinson (FR); Ioana Kloos, Rueil Malmaison (FR); Anne-Laure Sarry, Issy Les Moulineaux (FR); Sriram Balasubramanian, San Carlos, CA (US); Joseph Buggy, Mountain View, CA (US)

(21) Appl. No.: 14/130,134

(22) PCT Filed: Jul. 3, 2012
(86) PCT No.: PCT/FR2012/051540

§ 371 Date: Oct. 15, 2014

(30) Foreign Application Priority Data
Jul. 4, 2011 (EP) 11/02087

(51) Int. Cl. A61K 31/343; A61K 31/513; A61K 31/282; A61K 31/519

(52) U.S. Cl. CPC A61K 31/343; A61K 31/519; A61K 31/513; A61K 31/282
USPC 514/249

(57) ABSTRACT

Association between N-hydroxy-4-{2-[3-(N,N-dimethylaminomethyl)benzofuran-2-yl-carbonylamino]ethoxy}benzamide of formula (I):

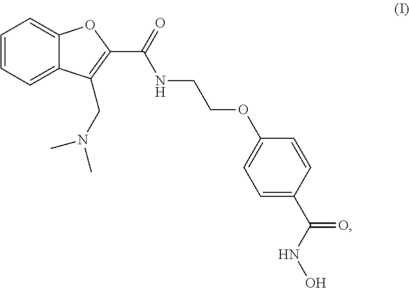 [SMILES: CN(C)Cc1c(C(=O)NCCOc2ccc(C(=O)NO)cc2)oc2ccccc12]

an addition salt thereof with a pharmaceutically acceptable acid or base, and FOLFOX.

Medicaments.

The present invention relates to a new association between N-hydroxy-4-{2-[3-(N,N-dimethylaminomethyl)benzofuran-2-ylcarbonylamino]ethoxy}benzamide of formula (I):

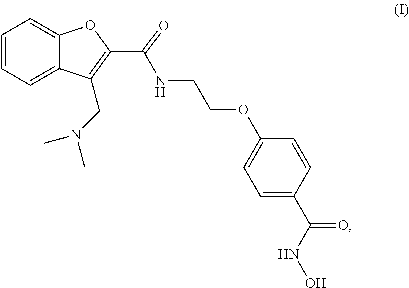 [SMILES: CN(C)Cc1c(C(=O)NCCOc2ccc(C(=O)NO)cc2)oc2ccccc12]

or an addition salt thereof with a pharmaceutically acceptable acid or base, and FOLFOX in the treatment of cancer, and more especially in the treatment of colorectal cancer, pancreatic cancer and gastric cancer in patients resistant to treatment based on FOLFOX.

N-hydroxy-4- {2-[3-(N,N-dimethylaminomethyl)benzofuran-2-ylcarbonylamino]ethoxy}-benzamide is a powerful histone deacetylase (HDAC) inhibitor described in patent application WO2004/092115. It allows inhibition of cell growth and induces apoptosis in cultured tumour cells in vitro, and it inhibits tumour growth in vivo in xenograft models (Buggy et al., Mol. Cancer Ther 2006 5(5) 1309). Its pharmacological profile makes it of major therapeutic value in the treatment of cancer.

The present invention relates to the association between N-hydroxy-4-{2-[3-(N,N-dimethylaminomethyl)benzofuran-2-ylcarbonylamino]ethoxy}benzamide of formula (I), or addition salts thereof with a pharmaceutically acceptable acid or base, and FOLFOX, as well as its properties for the treatment of cancer, and more especially for the treatment of colorectal cancer, pancreatic cancer and gastric cancer in patients resistant to treatment based on FOLFOX.

FOLFOX is a chemotherapy regimen used in the treatment of various cancers. It consists of the administration of oxaliplatin, folinic acid and 5-fluorouracil according to a schedule designed to optimise the efficacy of the treatment. It is commonly used in the treatment of colon cancer (De Gramont A. et al., J. Clin. Oncol. 2000 18 2938). Several phase II or III trials have moreover shown that FOLFOX may bring about a level of partial response in patients suffering from a gastric cancer (De Vita et al., Br. J. Cancer 2005 92 1644). In cancer of the pancreas, FOLFOX has also demonstrated efficacy as a second-line treatment subsequent to failure of gemcitabine therapy (Cascinu S. et al., Annals of Oncology 2010 21(Suppl 5) v55; Gebbia V. et al., Annals of Oncol 2007 18(Suppl 6) vi124; Li J. et al., J. Pancreas (Online) 2009 10(4) 361). However, the search for new therapeutic alternatives in oncology is still on-going. In particular, making patients who are resistant to clinically validated chemotherapies sensitive to those therapies constitutes a promising therapeutic strategy.

In an other embodiment of the invention, it has been shown that the effects of N-hydroxy-4-{2-[3-(N,N-dimethylaminomethyl)benzofuran-2-ylcarbonylamino]ethoxy}benzamide, or addition salts thereof with a pharmaceutically acceptable acid or base, make it possible to reverse the resistance to FOLFOX in patients who have previously been treated by that chemotherapy regimen.

This effect makes it possible to consider use of the association of N-hydroxy-4-{2-[3-(N,N-dimethylaminomethyl)benzofuran-2-ylcarbonylamino]ethoxy}benzamide and FOLFOX in the treatment of cancers first treated by FOLFOX in patients for whom, despite the treatment, disease progression has been observed, and more especially in the treatment of colorectal cancer, pancreatic cancer and gastric cancer in patients resistant to treatment based on FOLFOX.

In the context of the invention, preference is given to N-hydroxy-4-{2-[3-(N,N-dimethyl-aminomethyl)benzofuran-2-ylcarbonylamino]ethoxy}benzamide being used in the form of a hydrochloride salt.

In a preferred administration schedule, the compounds of the association according to the invention are administered over 5 consecutive days, that period being followed by 9 consecutive days without any administration. More especially, N-hydroxy-4-{2-[3-(N,N-dimethylaminomethyl)benzofuran-2-ylcarbonylamino]ethoxy}benzamide is administered for 4 consecutive days, whereas FOLFOX is administered from the third to the fifth day. Even more preferably, N-hydroxy-4-{2-[3-(N,N-dimethylaminomethyl)benzofuran-2-yl-carbonylamino]ethoxy}benzamide is administered for 4 consecutive days, whereas oxaliplatin and folinic acid are administered simultaneously on the third day, at the end of infusion of which 5-fluorouracil is administered continuously until the fifth day.

In a preferred embodiment, N-hydroxy-4-{2-[3-(N,N-dimethylaminomethyl)benzofuran-2-ylcarbonylamino]ethoxy}benzamide is administered in oral form.

The useful dosage varies according to the sex, age and weight of the patient, the administration route, and the nature of the cancer and of any associated treatments and ranges from 20 mg to 480 mg of N-hydroxy-4-{2-[3-(N,N-dimethylaminomethyl)-benzofuran-2-ylcarbonylamino]ethoxy}benzamide per day expressed in terms of the free base. As for the FOLFOX constituents, the doses administered in each 14-day cycle are:
85 mg/m² for oxaliplatin,
400 mg/m² for folinic acid,
2400 mg/m² for 5-fluorouracil.

### CLINICAL STUDY

A clinical study for testing the association of N-hydroxy-4-{2-[3-(N,N-dimethylamino-methyl)benzofuran-2-ylcarbonylamino]ethoxy}benzamide with FOLFOX is carried out on a maximum of 60 patients. Patients included in the study suffer from a colorectal cancer, a pancreatic cancer or a gastric cancer, and they have been treated by a treatment based on FOLFOX (i.e., alone or in association with other treatments) prior to their inclusion. More precisely, the study consists of 14-day cycles, each of the cycles being performed as follows: N-hydroxy-4-{2-[3-(N,N-dimethylaminomethyl)benzofuran-2-ylcarbonylamino]ethoxy}benzamide (in the form of a hydrochloride salt) is administered for 4 consecutive days whilst oxaliplatin and folinic acid are administered simultaneously on the third day, at the end of infusion of which 5-fluorouracil is administered continuously until the fifth day. Initially, the daily dose of N-hydroxy-4-{2-[3-(N,N-dimethylaminomethyl)benzofuran-2-ylcarbonylamino]ethoxy}benzamide hydrochloride is between 160 and 360 mg inclusive, in the form of two p.o. administrations 4 hours apart. As for the FOLFOX constituents, the doses administered in each cycle are:
85 mg/m² for oxaliplatin,
400 mg/m² for folinic acid,
2400 mg/m² for 5-fluorouracil.

At the end of a treatment cycle, the toxicity of the regimen associating N-hydroxy-4-{2-[3-(N,N-dimethylaminomethyl)benzofuran-2-ylcarbonylamino]ethoxy}benzamide and FOLFOX is assessed. If no prohibitive toxicity is observed, the patient continues the treatment. Every four cycles, the efficacy of the treatment is assessed by evaluating the tumour response (CT scan, MRI, etc.). The acceptability profile is also evaluated (especially haematologic and cardiac toxicity).

The results show that the use of N-hydroxy-4-{2-[3-(N,N-dimethylaminomethyl)benzofuran-2-ylcarbonylamino]ethoxy}benzamide in association with FOLFOX is promising for the treatment of cancer, and more especially for the treatment of colorectal cancer, pancreatic cancer and gastric cancer in patients resistant to treatment based on FOLFOX.

1-16. (canceled)

17. A method of treating cancer, in a subject in need thereof, comprising administration of an effective amount of the compounds including N-hydroxy-4-{2-[3-(N,N-dimethylaminomethyl)benzofuran-2-yl-carbonylamino]ethoxy}benzamide of formula (I): 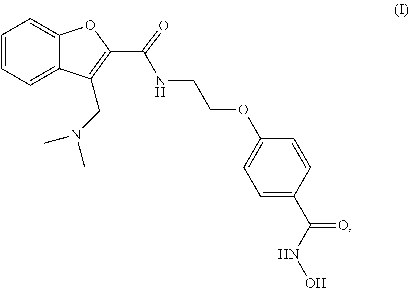 [SMILES: CN(C)Cc1c(C(=O)NCCOc2ccc(C(=O)NO)cc2)oc2ccccc12]
or an addition salt thereof with a pharmaceutically acceptable acid or base, in association with FOLFOX compounds and treatment.

18. The method of claim 17, wherein the N-hydroxy-4-{2-[3-(N,N-dimethylaminomethyl)benzofuran-2-ylcarbonylamino]ethoxy}benzamide compound is in the form of a hydrochloride salt.

19. The method of claim 17, wherein the cancer is colorectal cancer, and the subject is resistant to treatment based on FOLFOX.

20. The method of claim 17, wherein the cancer is pancreatic cancer, and the subject is resistant to treatment based on FOLFOX.

21. The method of claim 17, wherein the cancer is gastric cancer, and the subject is resistant to treatment based on FOLFOX.

22. The method of claim 17, wherein the compounds are administered over five consecutive days, that period being followed by nine consecutive days without any administration.

23. The method of claim 17, wherein the N-hydroxy-4-{2-[3-(N,N-dimethylaminomethyl)benzofuran-2-ylcarbonylamino]ethoxy}benzamide compound is administered for four consecutive days, and wherein the FOLFOX compounds are administered from the third to the fifth day.

24. The method of claim 17, wherein the N-hydroxy-4-{2-[3-(N,N-dimethylaminomethyl)benzofuran-2-ylcarbonylamino]ethoxy}benzamide compound is administered for four consecutive days, and wherein the FOLFOX compounds oxaliplatin and folinic acid are administered simultaneously on the third day at the end of infusion, and wherein the FOLFOX compound 5-fluorouracil is administered continuously until the fifth day.
